(19) United States
(12) Patent Application Publication
(10) Pub. No.: US 2016/0324456 A1
(43) Pub. Date: Nov. 10, 2016

(54) Methods and Systems for Obtaining Clinical Samples

(71) Applicant: Theranos, Inc., Palo Alto, CA (US)

(72) Inventors: Elizabeth A. Holmes, Palo Alto, CA (US); Pey-Jiun Ko, Palo Alto, CA (US)

(21) Appl. No.: 15/092,279

(22) Filed: Apr. 6, 2016

Related U.S. Application Data
(63) Continuation of application No. PCT/US2014/060539, filed on Oct. 14, 2014.
(60) Provisional application No. 61/890,870, filed on Oct. 14, 2013.
(60) Provisional application No. 61/894,166, filed on Oct. 22, 2013.

(51) Int. Cl. A61B 5/15; G01N 33/49; A63H 3/00; A61F 7/03; A61B 10/00

(52) U.S. Cl. CPC A61B 5/150839; A61F 7/03; A61B 5/150022; A61B 5/150122; A61B 10/0038; A61B 10/0048; A61B 10/0051; A61B 10/0058; A61B 10/0064; A61B 10/007; A61B 5/150038; A63H 3/003; G01N 33/49; A61F 2007/0036

(57) ABSTRACT

Methods for obtaining a sample from a subject include providing a sample collection room within a retail store; and obtaining a sample from the subject. Sample collection rooms may include one or more of a warming table; a warming plate; a fingertip warmer; an air-warmer; a reclining chair; a calming feature; and adjustable lighting. Samples may be analyzed at the retail location or sent to elsewhere for analysis. Sample collection rooms may house a sample analysis device or system. Samples may be small, e.g., a finger-stick, and may be analyzed in a short period of time, e.g., in less than five hours, or less than four hours.

### BACKGROUND

Clinical samples are useful in many situations, including being useful for monitoring the health of subjects, for diagnosing diseases or pathological conditions, and for monitoring the progress of therapeutic interventions, among various uses.

Obtaining clinical samples is required for such uses; however, subjects may find providing clinical samples, such as blood, urine, or tissue swab samples, to be painful, or difficult, or inconvenient. Accordingly, improves methods for obtaining clinical samples are needed.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

Improved methods for obtaining clinical samples include providing a designated location in a retail store where a clinical sample may be provided for analysis. In embodiments, improved methods for obtaining clinical samples include providing a designated location in a retail store, where the designated location is configured for obtaining a clinical sample; and obtaining a clinical sample from a subject. In embodiments, the sample may be analyzed at the same location; or at an adjacent location; or may be sent to a different location for analysis. In embodiments, the sample may be analyzed by an automatic analysis device or system at the same location; or at an adjacent location; or at a different location. A designated location in a retail store may be a sample collection room.

Applicants disclose methods for obtaining a sample from a subject, comprising providing a sample collection room in a retail store; and collecting a sample from a subject. Applicants disclose methods for obtaining a sample from a subject, comprising providing a sample collection room in a retail store; warming a fingertip, or finger, or hand of the subject; collecting a sample from a subject; and obtaining the sample. Applicants disclose methods for obtaining a sample from a subject, comprising providing a sample collection room in a retail store, the sample collection room comprising a warming table; warming a fingertip, or finger, or hand of the subject; and obtaining a sample from the subject. Applicants disclose methods for obtaining a sample from a subject, comprising providing a sample collection room in a retail store, the sample collection room comprising a chair; warming a fingertip, or finger, or hand of the subject; and obtaining a sample from the subject. Applicants disclose methods for obtaining a sample from a subject, comprising providing a sample collection room in a retail store, the sample collection room comprising a chair having a warming plate or other warming element; warming a fingertip, or finger, or hand of the subject; and obtaining a sample from the subject. Applicants disclose methods for obtaining a sample from a subject, comprising providing a sample collection room in a retail store, the sample collection room comprising an air-warmer; warming a fingertip, or finger, or hand of the subject; and obtaining a sample from the subject. Applicants disclose methods for obtaining a sample from a subject, comprising any of the foregoing methods, and providing the subject with a fingertoy, or placing a fingertoy on a finger of a subject following obtaining a sample from the subject. It will be understood that a step of warming a fingertip, or finger, or hand of a subject may comprise warming by use of a warming table, or warming plate, or air-warmer, or other warming device, or a combination thereof. It will be understood that the foregoing methods may be used together, and may be used in any combination.

In embodiments, the sample is a small sample. For example, in embodiments of the methods for obtaining a sample from a subject, the sample is obtained from a finger-stick. For example, in embodiments, the sample comprises a few drops, or two drops, or one drop, of blood obtained from a small lancet puncture in the skin of the subject. For example, in embodiments, the sample is a small volume sample of blood, or of urine, or of saliva, or of tears, or other bodily secretion or excretion. For example, in embodiments, the sample is a small sample having a volume of less than about 200 μL, or less than about 150 μL, or less than about 100 μL, or less than about 75 μL, or less than about 50 μL, or less than about 25 μL, or less.

Applicants disclose methods for analyzing a sample obtained from a subject, comprising providing a sample collection room in a retail store; collecting a sample from a subject; and analyzing the sample. Applicants disclose methods for analyzing a sample obtained from a subject, comprising providing a sample collection room in a retail store; warming a fingertip, or finger, or hand of the subject; collecting a sample from a subject; and analyzing the sample. Applicants disclose methods for analyzing a sample obtained from a subject, comprising providing a sample collection room in a retail store, the sample collection room comprising a warming table; warming a fingertip, or finger, or hand of the subject; collecting a sample from a subject; and analyzing the sample. Applicants disclose methods for analyzing a sample obtained from a subject, comprising providing a sample collection room in a retail store, the sample collection room comprising a chair; warming a fingertip, or finger, or hand of the subject; collecting a sample from a subject; and analyzing the sample. Applicants disclose methods for analyzing a sample obtained from a subject, comprising providing a sample collection room in a retail store, the sample collection room comprising a chair having a warming plate or other warming element; warming a fingertip, or finger, or hand of the subject; collecting a sample from a subject; and analyzing the sample. Applicants disclose methods for analyzing a sample obtained from a subject, comprising providing a sample collection room in a retail store, the sample collection room comprising an air-warmer; warming a fingertip, or finger, or hand of the subject; collecting a sample from a subject; and analyzing the sample. Applicants disclose methods for analyzing a sample obtained from a subject, comprising any of the foregoing methods, and providing the subject with a fingertoy, or placing a fingertoy on a finger of a subject following obtaining a sample from the subject. It will be understood that a step of warming a fingertip, or finger, or hand of a subject may comprise warming by use of a warming table, or warming plate, or air-warmer, or other warming device, or a combination thereof. It will be understood that the foregoing methods may be used together, and may be used in any combination.

In embodiments, the sample is analyzed at, or adjacent to, or near to, the sample collection room in the retail store. In embodiments, the sample is analyzed at a location other than at the retail store. In embodiments of the methods for analyzing a sample obtained from a subject, the sample is a small sample. For example, in embodiments, the sample is obtained from a finger-stick. For example, in embodiments, the sample comprises a few drops, or two drops, or one drop, of blood obtained from a small lancet puncture in the skin of the subject. For example, in embodiments, the sample is a small volume sample of blood, or of urine, or of saliva, or of tears, or other bodily secretion or excretion. For example, in embodiments, the sample is a small sample having a volume of less than about 200 μL, or less than about 150 μL, or less than about 100 μL, or less than about 75 μL, or less than about 50 μL, or less than about 25 μL, or less.

Accordingly, Applicants disclose methods for obtaining and analyzing a sample obtained from a subject, comprising: providing a sample collection room in a retail store; collecting a sample from a subject in the sample collection room; and analyzing the sample. Applicants disclose methods for obtaining and analyzing a sample obtained from a subject, comprising: providing a sample collection room in a retail store; collecting a small sample from a subject in the sample collection room; and analyzing the small sample. Applicants disclose methods for obtaining and analyzing a sample obtained from a subject, comprising: providing a sample collection room in a retail store; collecting a sample from a subject in the sample collection room; and analyzing the sample at the retail store location, or adjacent to the retail store location.

In embodiments, a sample collection room may include a comfortable chair or seating location for a subject, e.g., for use by a subject during collection of a sample from the subject. In embodiments, a comfortable chair may be a reclining chair, having an upright configuration and a reclined configuration. In embodiments, a comfortable chair may be a reclining chair having an upright configuration and a plurality of reclined configurations. In embodiments, a sample collection room may include a refrigerator, a cabinet, a sink, storage units, a work surface configured for writing, and other furniture and architectural elements.

In embodiments, a sample collection room may include a sample analysis device or system; such a sample analysis device or system may be an automated sample analysis device or system, or may be a semi-automated sample analysis device or system. In embodiments, a sample collection room may include a computer, computer accessories, communication equipment and capability effective to link a computer, cell phone, or other device with WiFi, internet, cable, or other connections.

Accordingly, Applicants disclose embodiments of systems for obtaining samples, and systems for analyzing samples, including at least the following.

A room in a retail location configured for obtaining a blood sample including warming table, a calming feature, and a chair, the room being configured for obtaining a clinical sample such as a blood sample. In embodiments, the chair is a reclining chair.

A room in a retail location configured for obtaining a blood sample including a warming table, a calming feature, a pass-through to an adjacent bathroom, and a reclining chair.

A room in a retail location configured for obtaining a blood sample including a warming table, a calming feature, adjustable lighting, a pass-through to an adjacent bathroom, and a reclining chair.

A room in a retail location configured for obtaining a blood sample including a warming table, a calming feature, adjustable lighting, calming scents, a pass-through to an adjacent bathroom, and a reclining chair.

A room in a retail location configured for obtaining a blood sample including a warming table, a calming feature, a calming image, calming sounds, adjustable lighting, calming scents, a pass-through to an adjacent bathroom, and a reclining chair.

A room in a retail location configured for obtaining a blood sample including a warming table, a calming feature, a calming image, calming sounds, calming colors, adjustable lighting, calming scents, a pass-through to an adjacent bathroom, and a reclining chair.

Applicants disclose methods for obtaining samples, and for analyzing samples, including the following methods.

A method for obtaining a sample from a subject, said subject having a body part, comprising: providing a sample collection room in a retail store; and obtaining a sample from said body part of said subject, wherein said obtaining is performed in said sample collection room.

A method for analyzing a sample obtained from a subject, said subject having a body part, comprising: providing a sample collection room in a retail store; obtaining, in said sample collection room, a sample from said body part of said subject; and analyzing the sample at or adjacent to the retail store.

A method for analyzing a sample obtained from a subject, said subject having a body part, comprising: providing a sample collection room in a retail store; obtaining, in said sample collection room, a sample from said body part of said subject; and analyzing the sample at or adjacent to the retail store within a short period of time after obtaining the sample.

A method for analyzing a sample obtained from a subject, said subject having a body part, comprising: providing a sample collection room in a retail store; obtaining, in said sample collection room, a sample from said body part of said subject; transporting the sample to an analysis location; and analyzing the sample at said analysis location within a short period of time after obtaining the sample. In embodiments, obtaining said sample comprises warming the body part of the subject. In embodiments, warming the body part comprises warming with a warming table. In embodiments, warming the body part comprises warming with a warming plate. In embodiments, warming the body part comprises warming with an air-warmer. In embodiments, the body part comprises a fingertip, a finger, a plurality of fingers, a hand, or other body part or body parts.

In embodiments of the foregoing methods, the methods may comprise warming a body part; for example, a body part may be warmed prior to obtaining a sample from the body part. In embodiments, warming of the body part comprises warming with a warming table. In embodiments, warming of the body part comprises warming with a warming plate. In embodiments, warming of the body part comprises warming with a fingertip warmer. In embodiments, warming of the body part comprises warming with an air-warmer. In embodiments, the body part comprises a fingertip, a finger, a plurality of fingers, a hand, or other body part or body parts.

In embodiments, systems and methods as disclosed herein may further include using a warming plate, or a fingertip warmer, or an air-warmer, or combination thereof. In embodiments, systems and methods as disclosed herein may further include using a fingertoy with or without a warmer therein. It will be understood that systems as disclosed herein may include combinations of some or all of the elements disclosed herein.

In one embodiment described herein, a novelty item is provided for use with a warmer comprising at least one structural element defining at least one area for receiving at least one warmer.

In another embodiment described herein, a novelty item is provided for use with a tissue warmer comprising at least one structural element defining at least one cavity for receiving at least one tissue warmer, wherein the at least one structural element is positioned within said novelty item along one surface of the novelty item that is configured to receive at least a portion of a subject being treated; at least a second structural element defining a toy portion of the novelty item.

In another embodiment described herein, a combination toy and finger warmer comprising a finger warmer device; a plurality of structural elements connectable to one another in a surrounding arrangement relative to the finger warmer device; where the plurality of structural elements comprise at least a plurality of first connector portions and a plurality of second connector portions, where all of the first connector portions have a first form and all of the second connector portions have a second form, such that any number of the structural elements can be joined with or without the warmer to form a toy assembly.

In another embodiment described herein, a combination novelty and finger warmer comprising: a plurality of pliable elements and a finger warmer, wherein the pliable elements are capable of assembly in surrounding engagement of the finger warmer; and where the plurality of pliable elements comprise at least a plurality of first connector portions and a plurality of second connector portions, where the plurality of pliable elements are detachable from the surrounding arrangement, such that any number of the pliable elements can be joined with or without the finger warmer to form a toy assembly.

In another embodiment described herein, a method for analyzing a sample obtained from a subject, said subject having a body part, comprising: providing a sample collection room in a retail store; warming a target tissue area on a subject with a combination novelty toy and warming device; obtaining, in said sample collection room, a sample from said body part of said subject; transporting the sample to an analysis location; and analyzing the sample at said analysis location within a short period of time after obtaining the sample.

It should be understood that any of the embodiments herein may be configured to one or more of the following features. In one non-limiting example, the method comprises giving only the novelty toy, but not the warming device, to the subject after the collection. Optionally, the warming of the body part comprises warming with a warming table; a warming plate; a fingertip warmer; an air-warmer; furniture for seating comprising a warming plate or other heating element; or a combination thereof. Optionally, the warming device is selected from a warming table, a warming plate, a chair or other furniture for seating comprising a warming plate, a fingertip warmer, and an air-warmer. Optionally, the warming device is configured to warm a body part of a subject effective to aid in the collection of a blood sample from said body part. Optionally, the target tissue is selected from a fingertip, a finger, and a hand. Optionally, the method comprises at least one technical feature from any of the prior descriptions. Optionally, the method comprises at least any two technical features from any of the prior descriptions. Optionally, the device comprises at least one technical feature from any of the prior descriptions. Optionally, the device comprises at least any two technical features from any of the prior descriptions. Optionally, the system comprises at least one technical feature from any of the prior descriptions. Optionally, the system comprises at least any two technical features from any of the prior descriptions.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIG. 1 shows a view of one embodiment as described herein.

FIGS. 2A and 2B show cross-sectional views of embodiments as described herein.

FIGS. 3A and 3B show side and cross-sectional views of one embodiment as described herein.

FIGS. 4A and 4B show side and cross-sectional views of one embodiment as described herein.

FIGS. 5A and 5B show side and cross-sectional views of one embodiment as described herein.

FIGS. 6 and 7 show side views of embodiments as described herein.

FIGS. 8A to 8E show views of embodiments a warmer as described herein.

FIG. 9 shows a view of an embodiment as described herein.

FIGS. 10A to 10E show views of embodiments of placement of a warmer as described herein.

FIG. 11 shows a view of an embodiment as described herein.

FIGS. 12A to 13D show views of embodiments as described herein.

### DETAILED DESCRIPTION

Methods, devices, and systems for obtaining a biological sample from a subject, such as, e.g., blood, are disclosed, for example, in U.S. Patent Application 61/875,092, filed Sep. 8, 2013; in U.S. Patent Application 61/852,484, filed Mar. 15, 2013; and in U.S. Patent Application 61/803,449, filed Mar. 19, 2013, the disclosures of all of which patent applications are hereby incorporated by reference in their entireties.

Description and disclosure of examples of obtaining samples and of sample analysis methods, devices, and systems, including automated analysis devices, semi-automated analysis devices, and systems comprising such devices, which may be used with the methods and systems disclosed herein may be found, for example, in U.S. Pat. No. 8,088,593; U.S. Pat. No. 8,380,541; U.S. patent application Ser. No. 13/769,798, filed Feb. 18, 2013; U.S. patent application Ser. No. 13/769,779, filed Feb. 18, 2013; U.S. patent application Ser. No. 13/769,820, filed Feb. 18, 2013; PCT/US2012/57155, filed Sep. 25, 2012; U.S. patent application Ser. No. 13/244,949, filed Sep. 26, 2011; U.S. Application Ser. No. 61/800,606, filed Mar. 15, 2013; U.S. Application Ser. No. 61/766,095, filed Feb. 18, 2013; and U.S. Application Ser. No. 61/673,245, filed Sep. 26, 2011; U.S. Patent Application 61/805,923, filed Mar. 27, 2013; in U.S. Patent Application 61/803,449, filed Mar. 19, 2013; in U.S. Pat. No. 8,088,593; in U.S. Pat. No. 8,380,541; in International Patent Application PCT/US2013/052141, filed Jul. 25, 2013; in U.S. Patent Application Ser. No. 61/879,664, filed Sep. 18, 2013; in U.S. Patent Application Ser. No. 61/837,167, filed Jun. 19, 2013; in U.S. Patent Application Ser. No. 61/874,976, filed Sep. 6, 2013; in U.S. Patent Application Ser. No. 61/766,116, filed Feb. 18, 2013; in U.S. Patent Application Ser. No. 61/802,194, filed Mar. 15, 2013; in U.S. patent application Ser. No. 13/244,947 filed Sep. 26, 2011; and in U.S. application Ser. No. 13/244,946, filed Sep. 26, 2011; the disclosures of all which patents and patent applications are hereby incorporated by reference in their entireties.

It is to be understood that both the foregoing general description and the following detailed description are exemplary and explanatory only and are not restrictive of the invention, as claimed. It may be noted that, as used in the specification and the appended claims, the singular forms “a”, “an” and “the” include plural referents unless the context clearly dictates otherwise. Thus, for example, reference to “a material” may include mixtures of materials, reference to “a compound” may include multiple compounds, and the like. References cited herein are hereby incorporated by reference in their entirety, except to the extent that they conflict with teachings explicitly set forth in this specification.

In this specification and in the claims which follow, reference will be made to a number of terms which shall be defined to have the following meanings:

“Optional” or “optionally” means that the subsequently described circumstance may or may not occur, so that the description includes instances where the circumstance occurs and instances where it does not. For example, if a device optionally contains a feature for a sample collection unit, this means that the sample collection unit may or may not be present, and, thus, the description includes both structures wherein a device possesses the sample collection unit and structures wherein sample collection unit is not present.

As used herein, the terms “substantial” means more than a minimal or insignificant amount; and “substantially” means more than a minimally or insignificantly. Thus, for example, the phrase “substantially different”, as used herein, denotes a sufficiently high degree of difference between two numeric values such that one of skill in the art would consider the difference between the two values to be of statistical significance within the context of the characteristic measured by said values. Thus, the difference between two values that are substantially different from each other is typically greater than about 10%, and may be greater than about 20%, preferably greater than about 30%, preferably greater than about 40%, preferably greater than about 50% as a function of the reference value or comparator value.

As used herein, the term “point of service location” (POS) may include locations where a subject may receive a service (e.g. testing, monitoring, treatment, diagnosis, guidance, sample collection, ID verification, medical services, non-medical services, etc.), and may include, without limitation, a subject's home, a subject's business, the location of a healthcare provider (e.g., doctor), hospitals, emergency rooms, operating rooms, clinics, health care professionals' offices, laboratories, retailers [e.g. pharmacies (e.g., retail pharmacy, clinical pharmacy, hospital pharmacy), drugstores, supermarkets, grocers, etc.], transportation vehicles (e.g. car, boat, truck, bus, airplane, motorcycle, ambulance, mobile unit, fire engine/truck, emergency vehicle, law enforcement vehicle, police car, or other vehicle configured to transport a subject from one point to another, etc.), traveling medical care units, mobile units, schools, day-care centers, security screening locations, combat locations, health assisted living residences, government offices, office buildings, tents, bodily fluid sample acquisition sites (e.g. blood collection centers), sites at or near an entrance to a location that a subject may wish to access, sites on or near a device that a subject may wish to access (e.g., the location of a computer if the subject wishes to access the computer), a location where a sample processing device receives a sample, or any other point of service location described elsewhere herein.

As used herein, a “sample”, or “biological sample”, or “clinical sample” refers to a sample of fluid, tissue, secretion, or excretion obtained from a subject. A clinical sample may be a sample of blood, serum, plasma, saliva, sputum, urine, gastric fluid, digestive fluid, tears, sweat, stool, semen, vaginal fluid, interstitial fluid, fluid derived from tumorous tissue, ocular fluids, mucus, earwax, oil, glandular secretions, spinal fluid, skin, cerebrospinal fluid from within the skull, tissue, fluid or material from a nasal swab, a throat swab, a cheek swab, or nasopharyngeal wash, biopsy fluid or material, placental fluid, amniotic fluid, cord blood, lymphatic fluids, cavity fluids, pus, microbiota obtained from a subject, meconium, breast milk, or other secretion or excretion. A sample may be a breath sample, a hair sample, a fingernail sample, or other sample.

Biological samples may include nasopharyngeal wash, or other fluid obtained by washing a body cavity or surface of a subject, or by washing a swab following application of the swab to a body cavity or surface of a subject. Nasal swabs, throat swabs, stool samples, hair, finger nail, ear wax, breath, and other solid, semi-solid, or gaseous samples may be processed in an extraction buffer, e.g., for a fixed or variable amount of time, prior to their analysis. The extraction buffer or an aliquot thereof may then be processed similarly to other fluid samples if desired. Examples of tissue samples of the subject may include but are not limited to, connective tissue, muscle tissue, nervous tissue, epithelial tissue, cartilage, cancerous sample, or bone. The sample may be obtained from a human or animal. The sample may be obtained from a vertebrate, e.g., a bird, fish, or mammal, such as a rat, a mouse, a pig, an ape, another primate (including humans), a farm animal, a sport animal, or a pet. The sample may be obtained from a living or dead subject. The sample may be obtained fresh from a subject or may have undergone some form of pre-processing, storage, or transport.

Thus, as used herein, a “sample” may be but is not limited to a blood sample, or a portion of a blood sample, may be of any suitable size or volume, and is preferably of small size or volume. In some embodiments of the assays and methods disclosed herein, measurements may be made using a small volume blood sample, or no more than a small volume portion of a blood sample, where a small volume comprises no more than about 5 mL; or comprises no more than about 3 mL; or comprises no more than about 2 mL; or comprises no more than about 1 mL; or comprises no more than about 500 μL; or comprises no more than about 250 μL; or comprises no more than about 100 μL; or comprises no more than about 75 μL; or comprises no more than about 50 μL; or comprises no more than about 35 μL; or comprises no more than about 25 μL; or comprises no more than about 20 μL; or comprises no more than about 15 μL; or comprises no more than about 10 μL; or comprises no more than about 8 μL; or comprises no more than about 6 μL; or comprises no more than about 5 μL; or comprises no more than about 4 μL; or comprises no more than about 3 μL; or comprises no more than about 2 μL; or comprises no more than about 1 μL; or comprises no more than about 0.8 μL; or comprises no more than about 0.5 μL; or comprises no more than about 0.3 μL; or comprises no more than about 0.2 μL; or comprises no more than about 0.1 μL; or comprises no more than about 0.05 μL; or comprises no more than about 0.01 μL.

As used herein, a “small volume” refers to a volume of less than about 1 mL, or less than about 500 μL, or less than about 250 μL, or less than 150 μL, or less than about 100 μL, or less than about 50 μL, or less than about 25 μL, or less. In particular embodiments, a small volume, such as a “finger-stick” volume, may comprise less than about 250 μL, and typically comprises less than 150 μL, or less than about 100 μL, or less than about 50 μL, or less than about 25 μL, or less.

As used herein, a “short period of time” refers to a period of time of about 5 hours or less, or about 4 hours or less, or about 3 hours or less, or about 2 hours or less, or about 1 hour or less, or about 50 minutes or less, or about 40 minutes or less, or about 30 minutes or less, or about 20 minutes or less, or about 10 minutes or less, or about 5 minutes or less. A short period of time may be determined with respect to an initial time; the initial time may be the time at which a sample analysis began; the initial time may be the time at which a sample is inserted into a device for the analysis of the sample; the initial time may be the time at which a sample was obtained from a subject.

It should be understood that embodiments in this disclosure may be adapted to have one or more of the features described below.

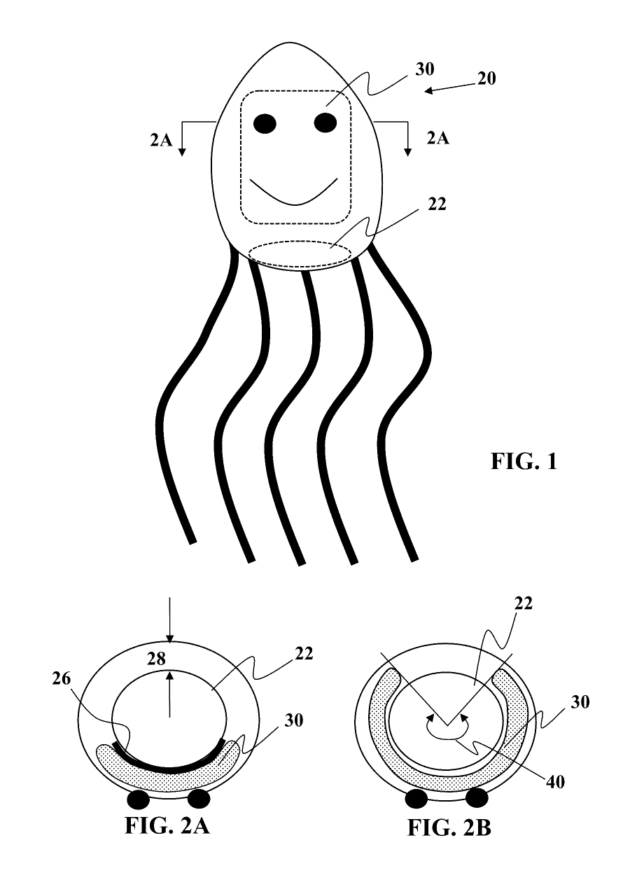

Referring now to FIG. 1, one embodiment of fingertip warmer will now be described. A fingertip warmer can be useful to increase blood flow to that area, which may be a target area for blood extraction. In some embodiments of methods for obtaining a blood sample from a subject, blood may be obtained from a small skin puncture at the site to be warmed. Such a small skin puncture may be, e.g., a small skin puncture on a finger, such as on a fingertip; a small skin puncture on a toe, such as on or near a tip of a toe; a small skin puncture on a foot, such as on or near the heel; or other small skin puncture. A small skin puncture is on a finger may be termed a “finger-stick”; the term finger-stick may also be used to describe the volume of a sample, i.e., a finger-stick volume is one, two, or a few drops (e.g., about 50 μL to about 300 μL, or about 75 μL to about 250 μL, or about 80 μL to about 150 μL).

FIG. 1 shows one embodiment of a fingertip warmer 20 wherein the exterior has a likeness of an animal such as but not limited to a jellyfish. This exterior form may make the blood extraction process proceed more smoothly for pediatric subjects. The toy-like fingertip warmer 20 can be both a useful distraction to take the pediatric subjects attention away from the procedure and/or it can be a reward for cooperating with the blood or other sample extraction procedure. Although this non-limiting example uses a jellyfish for fingertip warmer 20, other animals such as a squid, a fish, a bears, a pig, or others are not excluded. Non-animal toy-like exterior appearance for the fingertip warmer 20 can also be used such as but not limited to a rocket, an airplane, a car, a race car, a train, a train locomotive, a ship, a space ship, or other toy-like shape. Some may form the exterior of plush material that provides a stuffed animal type feel. Optionally, others may use a combination of hard and soft materials. Optionally, still others may use primarily hard materials for forming the toy-like exterior.

FIG. 1 shows that fingertip warmer 20 includes at least one opening 22 for receiving at least one portion of a finger such as but not limited to a fingertip therein. Optionally, some embodiments may have opening 22 sized for at least two fingers. Optionally, some embodiments may have multiple openings 22, each sized for at least one finger. Optionally, some embodiments may be configured to warm other portion or portions of the finger and not necessarily the fingertip.

FIG. 1 also shows that this embodiment includes at least one pouch 30 sized to receive at least one heat providing object. In one non-limiting example, the pouch 30 is positioned on the same side as the face or other primary-interest feature of the finger-tip warmer. In this manner, the pouch 30 which is sized to hold at least a portion of at least one heat providing object is positioned to be over at least a portion of the pad portion of the fingertip. In this manner, the tissue to be targeted which is typically not on the side of the finger nail, can be heated to a desired temperature to increase blood flow. This pad portion of the fingertip is typically on the palm side of the finger and may include one or more sides of the fingertip. In at least one embodiment, pouch 30 is openable from only one end and this opening can be a closable opening. It should be understood that the closable opening can be closed by closure devices such as but not limited to velco, hook and fabric closure device, button, zipper, ziplock, plastic zipper, sliding plastic zipper, or other closure device that may be developed in the future. The pouch 30 may have one or more closure devices which may be on the same or different edge(s) of the pouch. Although pouch 30 is shown as a rectangle, it should be understood that other shapes such as but not limited to polygonal, circular, triangular, square, oval, single or multiple combinations of shapes, or other shapes. In embodiments, the shape of the heat providing object to be contained in the pouch 30 may have the same shape as the pouch 30 or optionally, some embodiments have heat providing object(s) of different shape than the pouch 30.

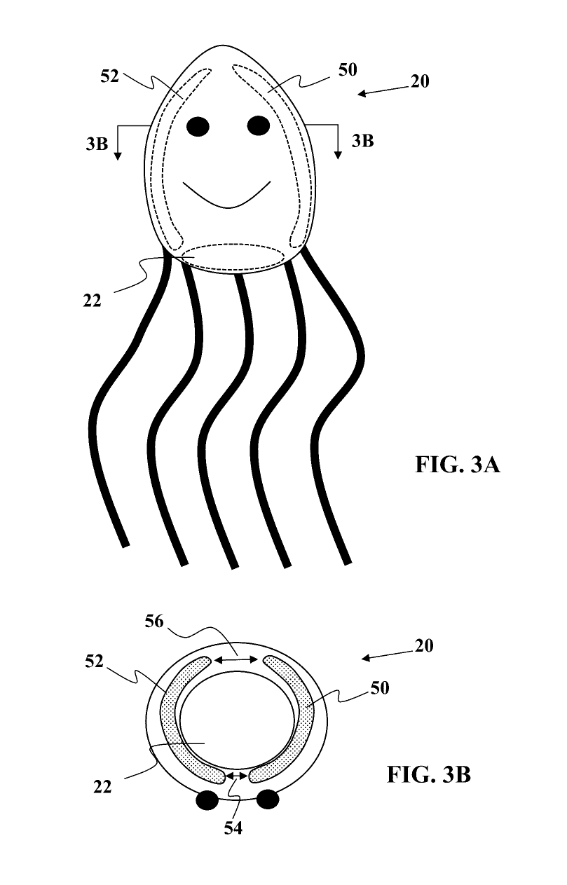

Referring now to FIG. 2A, a cross-sectional view if shown of the fingertip warmer 20. In this embodiment, a pouch 30 when viewed from top-down, is mostly on only one side of the fingertip warmer 20. As seen in FIGS. 1 and 2A, this embodiment has at least a majority portion of the pouch 30 is positioned on the side of the fingertip warmer 20 showing the interest feature, which in this embodiment is the face of the jellyfish. The alignment of the pocket 30 on the “face-side” of fingertip warmer 20 can be useful as the user, particularly a pediatric subject, will likely put the face side toward the user which also positions the pouch 30 and the heat providing object therein on the target tissue, which in this non-limiting example is the pad side of a user's fingertip. Thus, more generally, the main attraction feature of the exterior of the fingertip warmer 20 can have a pre-determined orientation relative to the pouch 30. This can also extend to the orientation that the user may place the warmer on the user's finger. By way of non-limiting example, the opening 22 can lead to a cavity that is contoured or otherwise configured to fit more preferentially in one orientation over the user's finger. This alignment of primary exterior feature and the pouch 30 holding the heat providing object(s) can facilitate the positioning of heat to the desired target tissue. For any of the embodiments herein, it should be understood that some embodiments may include at least one sanitary barrier layer that may be coupled to surface area of the novelty item where a subject's finger may be in contact with the surface. This layer may be removable or non-removable.

FIG. 2A also shows that for at least some embodiments herein, there may be a reduced thickness lining 26 to position the element in the pouch 30 closer to the target tissue than if the pouch were coupled to the target tissue through the thickness of the wall of the warmer as indicated by arrow 28. Optionally, some embodiments may use a patch of material that is different from the material used in the rest of the warmer liner, wherein the material of lining 26 is selected for improved thermal conductivity. Optionally, the material of lining 26 is merely thinner than the other material of the warmer. Optionally, the material at portion 26 is the same as other material in the lining except there is reduced padding in the area that may be present in other areas of the warmer 20.

As seen in FIG. 2B, at least some embodiments can the pouch 30 span more than 180° arc 40 in terms of coverage around an object in pouch 22. In one embodiment, the size of the pouch 30 and the heat providing element therein spans at least a 180° arc around the finger or other tissue in the opening 22. Optionally, the size of the pouch 30 and the heat providing element therein spans at least a 170° arc around the finger or other tissue in the opening 22. Optionally, the size of the pouch 30 and the heat providing element therein spans at least a 160° arc around the finger or other tissue in the opening 22. Optionally, the size of the pouch 30 and the heat providing element therein spans at least a 150° arc around the finger or other tissue in the opening 22. Optionally, the size of the pouch 30 and the heat providing element therein spans at least a 170° arc around the finger or other tissue in the opening 22. In this manner, more of the tissue is heated and this also increases the likelihood that the target tissue is heated. Optionally, this also increases the surface area of the tissue being heated which can also be beneficial in terms

In embodiments, the heat providing object is used to bring the target tissue to between about 40° C. to about 50° C. In embodiments, the heating brings target tissue to within a temperature range of between about 40° C. to about 44° C. In embodiments, the heating brings target tissue to within a temperature range of between about 41° C. to about 43° C. In embodiments, the heating brings target tissue to a temperature of about 42° C. In embodiments, the heating brings target tissue to within a temperature range of between about 44° C. to about 47° C. In one embodiment, the temperature is sufficient to increase blood flow to yield 120 uL of sample. In one embodiment, the temperature is sufficient to increase blood flow to yield 130 uL of sample. In one embodiment, the temperature is sufficient to increase blood flow to yield 140 uL of sample. In one embodiment, the temperature is sufficient to increase blood flow to yield 150 uL of sample. Optionally, the thermally controlled site is a shaped surface is contoured to match that of the target site on a patient. Optionally, the size of the pouch 30 holding the heat providing object is sized to be greater than the area of the target tissue.

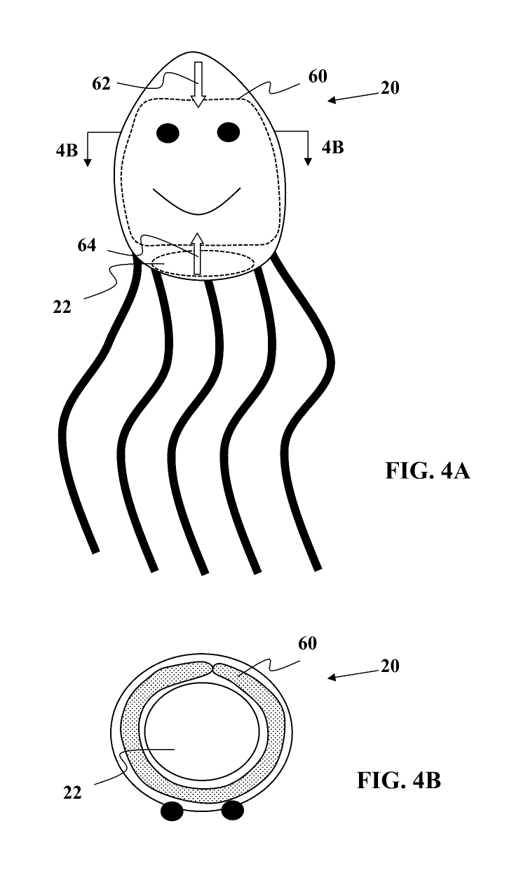

Referring now to FIG. 3A, a still further embodiment of a fingertip warmer 20 is shown. This embodiment shows that there may be two pouches 50 and 52 in the warmer 20. As seen in FIG. 3A, the pouches 50 and 52 can position the heat providing objects on opposing sides of the finger.

FIG. 3B shows a top-down view where it can be seen that in this particular embodiment, the pouches 50 and 52 are configured to more fully heat the tissue oriented along the “face” side of the warmer 20 as noted by the smaller gap 54 between the pouches relative to the opposing gap 56. It should be understood that the pouches 50 and 54 can have openings to allow for insertion of the heat providing object(s). These openings can be access from above, below, or from the sides of the warmer 20. This can be by way of openings on the interior or exterior of the warmer 20. This embodiment can also be useful for heating more of the sides of the pad portion of finger.

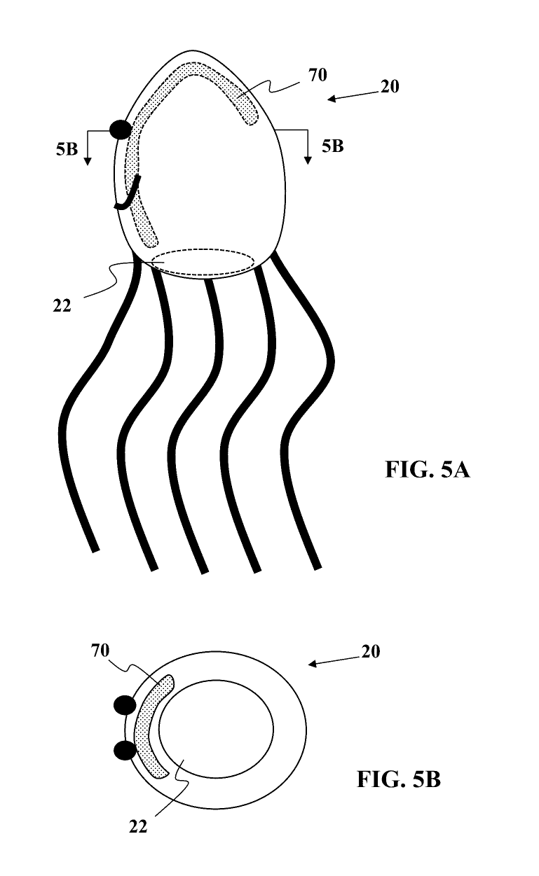

Referring now to FIG. 4A, a still further embodiment of a fingertip warmer 20 is shown. This embodiment shows a pouch 60 that is configured to fully or substantially circumscribe the circumference of the opening 22. This pouch 60 can be assessable from the top and/or bottom of an interior and/or exterior of the warmer 20 as indicated by arrows 62 and 64. This embodiment of pouch 60 can be configured to heat the target tissue portion of the fingertip.

As seen in FIG. 4B, the ends of the pouch 60 can touch. Optionally, some embodiments can have the pouch 60 be a circumferentially continuous pocket when viewed top down. In some embodiments, even though the pouch 60 is circumferentially continuous, the heat providing element can be a rolled element having a first circumferential end and a second circumferential end. Optionally, some may have a ring or other circumferentially continuous shape. Optionally, some embodiment can use a plurality of discrete heating elements in the pouch 60, such elements being flexibly connected together or not connect at all.

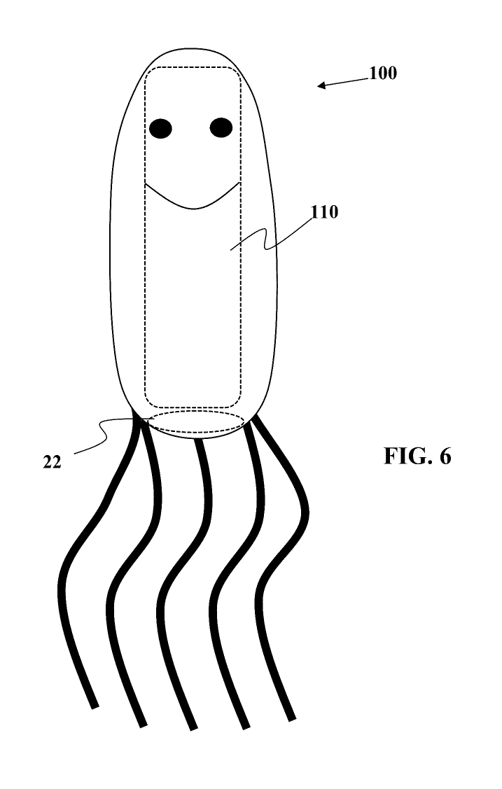

Referring now to FIG. 5A, a still further embodiment of a fingertip warmer 20 is shown. FIG. 5A shows a side view wherein the pouch 70 is shown extending along a primary interest portion of the warmer 20 and then has a portion that extends over a distal end of the warmer 20 and in this embodiment, wraps a portion of the other, opposing side of the warmer 20. In this manner, this warmer 20 will heat at least a pad portion of the finger and at least a distal tip of the finger.

FIG. 5B shows that this particular example uses a pouch 70 that is sufficiently wide to cover at least a pad portion of the width of the finger and this extends to cover at least the distal tip of the finger. Optionally, other embodiments may have a pouch 70 wider than the finger. Optionally, other embodiments may have a pouch 70 narrower than the width of the finger. For any of the embodiments herein, it should be understood that some embodiments may include at least one sanitary barrier layer that may be coupled to surface area of the novelty item where a subject's finger may be in contact with the surface. This layer may be removable or non-removable.

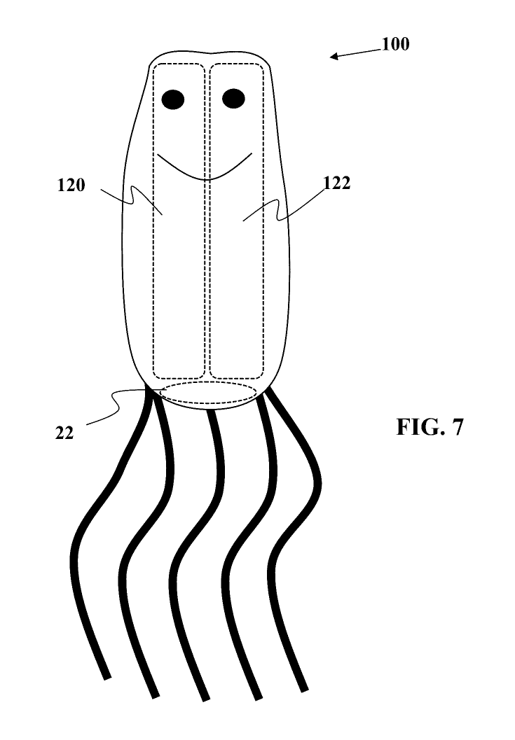

Referring now to FIG. 6, a still further embodiment of a fingertip warmer 100 is shown. This embodiment shows that the warmer 100 is sized to have a length that covers more than one phalanx. A subject may have a hand comprising a plurality of digits such as a thumb, index finger, middle finger, ring finger, or small finger. A digit may comprise a proximal phalanx, an intermediate phalanx, and a distal phalanx. A hand may have at two primary sides: a palm side and a fingernail side. The embodiment of FIG. 6 can have a pouch 110 that covers at least the distal phalanx and the intermediate phalanx. Optionally, one embodiment of pouch 110 can cover the distal phalanx, the intermediate phalanx, and at least a portion of the proximal phalanx. This circumferential coverage can one of the any of the configurations shown in various embodiments described herein and is not limited to the coverage shown in FIG. 6.

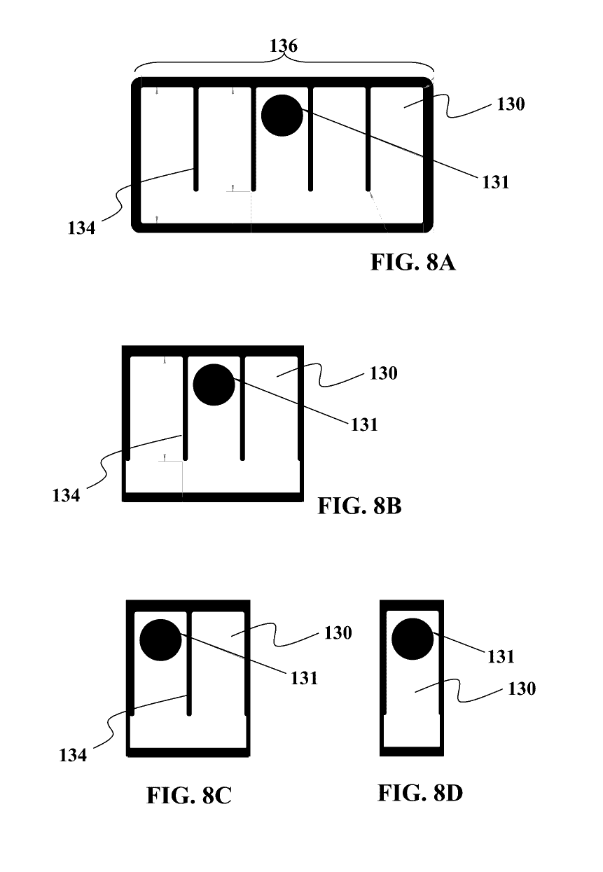

Referring now to FIG. 7, a still further embodiment of a finger warmer 100 is shown. This embodiment shows that the warmer 100 is sized to have a length that covers more than one phalanx. A subject may have a hand comprising a plurality of digits such as a thumb, index finger, middle finger, ring finger, or small finger. A digit may comprise a proximal phalanx, an intermediate phalanx, and a distal phalanx. A hand may have at two primary sides: a palm side and a fingernail side. The embodiment of FIG. 6 can have a pouch 120 that covers at least the distal phalanx and the intermediate phalanx. Optionally, one embodiment of pouch 120 can cover the distal phalanx, the intermediate phalanx, and at least a portion of the proximal phalanx. This circumferential coverage can one of the any of the configurations shown in various embodiments described herein and is not limited to the coverage shown in FIG. 7. As seen in FIG. 7, this embodiment can have at least a second pouch 122 for receiving another thermal control object. This can be a heating or a cooling object. The pouch 122 can be the same length as the pouch 120. Optionally, it may be shorter than pouch 120. Optionally, it may be longer than pouch 120. As seen in FIG. 7, that are oriented to each cover a portion of the exterior area of interest of the warmer 100. Other embodiments may orient the pouches in other alignments or orientations on the warmer 100.

In embodiments, the thermal controlled surface of a warmer may be or include, for example, plastic, hard rubber, acrylic, polymer, and other materials. In embodiments, the thermal controlled site comprises a soft or padded surface that is easily cleaned, and that may be sterilized. Such a surface may be or include, for example, cloth, rubberized cloth, soft rubber, and other materials.

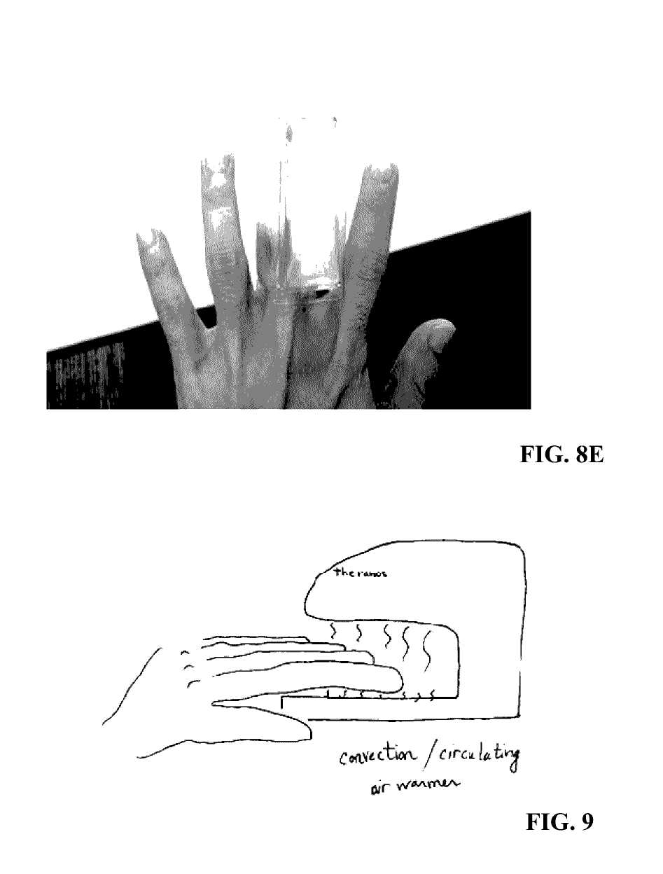

FIG. 8A shows a schematic view of an example of a finger warmer having features as disclosed herein, providing a top view of a finger warmer illustrating some design features and exemplary dimensions. FIG. 8B shows a schematic example of a finger warmer having features as disclosed herein, but of reduced size and having only three “compartments” 130 instead of the five shown in FIG. 8A. FIGS. 8C shows an embodiment of a finger warmer with only two compartments. FIG. 8A shows a single compartment type finger warmer. As seen in the embodiments herein, a triggering device 131 is included in the finger warmer. Optionally, some embodiments may use a design wherein heating occurs upon mixing of two or more components, without a trigger 131. As seen in the figures, the baffles 134 may be used to facilitate folding of the warmer around an object. By way of example and not limitation, at least two of the compartments 130 remain interconnected as the baffles 134 do not completely separate one compartment 130 from fluid communication with another compartment 130. Optionally, the width of at least one compartment 130 is at least that of one finger width.

Optionally as seen in FIGS. 8A to 8E, a finger warmer having features as disclosed herein comprises a closed container enclosing a heating composition which can be triggered to provide heat. Thus, a finger warmer may comprise an enclosed compartment, having a flexible outer covering (e.g., a “skin”), such as a polymer, plastic, rubber, cloth, or other flexible material, enclosing materials which may, when desired, react in an exothermic reaction effective to release heat which may be used to warm an extremity of a subject. The flexible outer covering, or an inner portion of such a covering, is typically suitable for containing the enclosed materials without leakage, and is typically suitable for containing the enclosed materials in a state which prevents the occurrence of the exothermic reaction until it is purposefully triggered. Such an outer covering may be a water-proof covering, may be an air-tight covering, and, in embodiments, may be both water-proof and air-tight.

An outer covering of a finger warmer as disclosed herein is typically soft, or flexible, or both, and is typically configured to allow bending, folding, or other manipulation effective, e.g., to wrap a finger warmer around a finger, or around two or more fingers, of a subject. In embodiments, an outer covering of a finger warmer may comprise a polymer, such as Nylon®, Dacron®, Perlon®, Terylene®, Kevlar®, and other polymers or polymer blends. In embodiments, an outer covering of a finger warmer may comprise polyvinylchloride, polyethylene, polyurethane, or other polymer. In embodiments, an outer covering of a finger warmer may comprise a metallocene polymer, or a metallocene blend, such as, e.g., a metallocene nylon blend. In embodiments, a finger warmer may comprise a metallocene blend/nylon/metallocene blend composite. The perimeter of the outer covering, and the regions shown as vertical lines within the perimeter, may be fused together, e.g., by heat, or glue, or ultrasonic welding, or other means. In embodiments, a finger warmer may comprise an internal baffle, or a plurality of internal baffles, or may comprise two or more enclosed compartments. In embodiments, a finger warmer may be foldable, e.g., may be folded around an extremity of a subject, or folded around itself (folded up, as for storage). In embodiments, a finger warmer may be configured to be rolled, e.g., may be rolled around an extremity of a subject, or rolled around itself (e.g., rolled up, as for storage). In embodiments, a finger warmer may be flexible. In embodiments, a finger warmer may comprise hinges. As shown in FIG. 8A in an extended configuration, the finger warmer may have a substantially rectangular shape; the vertical lines within the rectangular perimeter indicate positions configured for folding the finger warmer. In embodiments, a finger warmer in an extended configuration may have a square, or round, or oval, or other shape.

In at least some embodiments, a finger warmer may have a circumferential perimeter 136 that may be roughly rectangular in shape, with a length 12 of between about 3 to about 12 inches, or between about 4 to about 10 inches, or between about 4.5 and about 8 inches, or between about 5 and about 7 inches. A perimeter 126 may enclose an inner perimeter. In embodiments, a finger warmer may have a length 12 of about 6 inches. A finger warmer may have a width 14 of between about 1 and about 6 inches, or between about 2 and about 5 inches, or between about 2.5 and about 4 inches. In embodiments, a finger warmer may have a width 14 of about 3 inches. A finger warmer may include an outer seam, or weld, which may have a seam width 24 of between about 0.05 inches to about 0.5 inches, or between about 0.1 inches to about 0.3 inches, or of about 0.2 inches. A finger warmer may thus provide an inner compartment 130, within the outer seam, having an internal width of between about 0.5 and about 5 inches, or between about 1 and about 4 inches, or between about 1.5 and about 3 inches, or of about 2.5 inches.

A finger warmer 10 may include an internal baffle 134 or multiple internal baffles 134, which may facilitate folding a finger warmer by providing a thinner, or lower-resistance portion than other portions of the finger warmer. Internal baffles 134 may extend between one wall of the perimeter and an opposite wall, or, an internal baffle 134 may extend partially from one wall, stopping short of the opposite wall of perimeter. A first internal baffle 134 may be spaced apart from a second internal baffle 134, for example, by a spacing 36 of between about 0.3 to about 2 inches, or of between about 0.5 to about 1.5 inches, or of about 1 inch. An internal baffle 134 may have a baffle width of between about 0.01 to about 0.5 inches, or between about 0.05 to about 0.2 inches, or may have a baffle width 32 of about 0.1 inches. An internal baffle 134 may have a baffle length of between about 0.5 to about 6 inches, or may have a baffle length 34 of between about 1 to about 4 inches, or between about 1.5 to about 3 inches. In embodiments, an internal baffle 134 may have a baffle length of about 2 inches.

In embodiments, a finger warmer provides an enclosed space (e.g., internal volume defined by the compartments 134). In embodiments, an internal compartment may comprise a volume of between about 5 mL to about 250 mL; or between about 10 mL and about 200 mL; or between about 15 mL and about 100 mL; or between about 20 mL and about 80 mL; or between about 30 mL and about 60 mL; or between about 35 mL and about 45 mL.

An enclosed space of a finger warmer may hold a heating composition (e.g., a composition capable of providing heat, by, for example, an exothermic reaction). For example, the enclosed space of a finger warmer may hold a supersaturated salt solution (e.g., supersaturated sodium acetate in water) containing a trigger element, such as a metal disk which, when flexed, triggers crystallization of the salt, thereby releasing heat. In embodiments, the enclosed space of a finger warmer may hold a water-based solution including iron (typically as a powder), typically with a salt, and often also with activated carbon and other materials; the iron, when exposed to air, releases heat as the iron is oxidized, effective to warm a subject or a subject's extremity.

A finger warmer may contain an oxidizable composition (e.g., a composition containing iron particles); or other composition which may release heat under proper conditions. An oxidizable composition may be contained or stored so as to prevent contact between the composition and air (or more particularly, between the composition and oxygen in the air); providing access to air initiates the desired heat generation. Thus, a finger warmer 10 containing an oxidizable composition within an internal compartment may be maintained ready for use by preventing contact between the oxidizable composition and air, and heating may be initiated by allowing contact between the oxidizable composition and air (e.g., by removing a covering over a port which allows contact between the oxidizable composition and air, or otherwise breaching an air-tight enclosure containing the oxidizable composition).

A finger warmer may contain a super-saturated solution, such as a super-saturated salt solution, which may release heat under proper conditions. A super-saturated salt solution (such as super-saturated sodium acetate) may include therein a trigger 131. A trigger 131 may be, for example, a metal disk which upon deformation is able to trigger the release of heat from the super-saturated solution. In embodiments in which a finger warmer has a single internal compartment, activation of heating composition within the internal compartment eventually activates all of the heating composition. In embodiments, such as embodiments in which an internal baffle 134, or multiple internal baffles 134, extend from one wall of perimeter to an opposite wall of perimeter to isolate one portion of internal compartment from another portion of internal compartment, a finger warmer may have separate portions of a heating composition enclosed in separate internal compartments. In such embodiments, in which a finger warmer has multiple internal compartments, activation of heating composition within one internal compartment may not activate the heating composition in another internal compartment, or may not activate the heating composition in all of the internal compartments.

In FIG. 8A, a trigger 131 is shown in place within the internal compartment. A trigger 131 is used to initiate the release of heat from a heating composition within a finger warmer 10 (such initiation may be termed “activation” of the heating composition). For example, flexing or deformation of a trigger 131 within a super-saturated salt solution may be effective to initiate salt crystallization in the super-saturated solution; when the salt is an appropriate salt, such crystallization generates heat. For example, deformation of a trigger 131 within a super-saturated sodium acetate solution is able to trigger crystal formation in that solution. Without being bound by theory, it is believed that sodium acetate crystals formed in cracks, on peaks or discontinuities, or on the surface of the trigger 131 may be dislodged by deformation of the trigger 131, and so made available as nucleation centers for further sodium acetate crystal formation. In embodiments, a trigger 131 may be a packet or container holding sodium acetate (or other salt) crystals, and be able to initiate release of heat from a surrounding super-saturated sodium acetate (or other salt) solution by release of the crystals or other contact between the crystals and the super-saturated solution (e.g., by tearing or otherwise opening the packet or container).

In embodiments, a trigger 131 may comprise a metal disk, e.g., a thin metal disk having a thickness of between about 0.05 to about 0.4 inches, or between about 0.1 to about 0.3 inches, or of about 0.2 inches. In embodiments, a trigger 131 may comprise a thin metal disk having a diameter of between about 0.1 to about 2 inches, or of between about 0.3 to about 1.5 inches, or of between about 0.5 to about 1 inches, or of about 0.75 inches. In embodiments, a trigger 131 is typically made of a material which does not degrade or decay when in contact with sodium acetate or other salt. In embodiments a trigger 131 may be a stainless steel disk; in alternative embodiments, a trigger 131 may be made of another metal, or other material able to trigger an exothermic reaction when flexed or broken. In embodiments, a trigger 131 may have a square, or rectangular, or oval, or other shape. In embodiments, a trigger 131 may have a continuous surface, or may have holes, or pores, or other features breaking up the continuity of the trigger surface. In embodiments, a trigger 131 may have a flat surface, or may have an irregular surface, or a rough surface, or may include grooves, depressions, ridges, elevations, or other features rising from, or dropping into, the surface of a trigger 131.

Prior to activation of a heating composition in the internal compartment of a finger warmer 10, the finger warmer 10 and its contents are typically at room temperature. Following activation of a heating composition, the finger warmer 10 and its contents may reach temperatures of between about 32° C. to about 48° C., or between about 35° C. to about 45° C., or between about 37° C. to about 43° C., or between about 39° C. to about 41° C. Heat generation to these levels may last for about 5 to 15 minutes, or about 8 to 10 minutes, following initiation of the heating process. If desired, the temperature range may be increased by alteration of the heating composition (e.g., by increasing the concentration of sodium acetate in a super-saturated sodium acetate solution, or by increasing the amount of concentration of iron in heating composition that relies on oxidation of iron to produce heat, etc.). Alternatively, lowering the concentration of salt or iron, or other active component of the heating composition may reduce the temperature range if less heat generation is desired.

FIG. 8A shows examples of a finger warmer which may be used to warm a subject's finger (or other extremity, e.g., a toe, or a heel) in order to enhance blood flow from the extremity during acquisition of a biological sample from the subject. A finger warmer having features as illustrated in FIG. 8 may be, for example, activated to provide warmth and wrapped around a finger (or two or more fingers). Following a minute, or two, or more time after wrapping, the sample may be acquired. For example, following a few minutes of warming, the finger or fingers of the subject may be unwrapped sufficiently to expose a fingertip, and a lancet applied so as to pierce the skin and allow a few droplets of blood to escape the fingertip and be collected as a biological sample.

FIG. 8A shows one embodiment of a heat providing object having features as disclosed herein comprises a closed container enclosing a heating composition which can be triggered to provide heat. In embodiments, a heat providing object may comprise a metallocene blend/nylon/metallocene blend composite. The perimeter and the regions shown as vertical lines within the perimeter may be fused together, e.g., by heat, or glue, or ultrasonic welding, or other means. In embodiments, a heat providing object may comprise a baffle, or plurality of baffles, or may comprise two or more enclosed compartments. In embodiments, a heat providing object may be foldable, e.g., may be folded around an extremity of a subject, or folded around itself (folded up, as for storage). In embodiments, a heat providing object may be configured to be rolled, e.g., may be rolled around an extremity of a subject, or rolled around itself (rolled up, as for storage). Before being wrapped around a finger, the heat providing object may have a substantially rectangular shape; the vertical lines within the rectangular perimeter indicate positions configured for folding the finger warmer.

In embodiments, a heat providing object provides an enclosed space (e.g., internal compartment) which holds a heating composition; as discussed above, a heating composition may include, for example, a super-saturated salt solution (such as super-saturated sodium acetate); an oxidizable composition (e.g., a composition containing iron particles); or other composition which may release heat. A super-saturated solution may include a trigger, such as a metal disk which, upon deformation, is able to trigger the release of heat from the solution. An oxidizable composition may be contained or stored so as to prevent contact between the composition and air (or more particularly, between the composition and oxygen in the air); providing access to air initiates the desired heat generation. A heat providing object may have a single internal compartment (since, in this illustration, baffle length is less than internal width, so that baffles do not completely isolate individual portions of internal compartment). In embodiments in which heat providing object has a single internal compartment, activation of heating composition within the internal compartment eventually activates all of the heating composition. In embodiments, such as embodiments in which a baffle, or multiple baffles, extend from one wall of perimeter to an opposite wall of perimeter to isolate one portion of internal compartment from another portion of internal compartment, a heat providing object may have separate portions of a heating composition enclosed in separate internal compartments. In such embodiments, in which a heat providing object has multiple internal compartments, activation of heating composition within one internal compartment may not activate the heating composition in another internal compartment, or may not activate the heating composition in all of the internal compartments.

FIG. 8E shows a heat providing object wrapped around the finger of a subject. The a heat providing object has been folded along baffles, and, as it is flexible, is also curved or flexed in other locations as well. Such folds, and such curvature, whether alone, or whether together, aid in wrapping a heat providing object around a finger, or portion thereof, of a subject, and aid in warming the finger, or portion thereof, of the subject in order to facilitate acquisition of a biological sample from the subject. In some embodiments, the heat providing object is inserted into the warmers 20 or 100 as described herein.

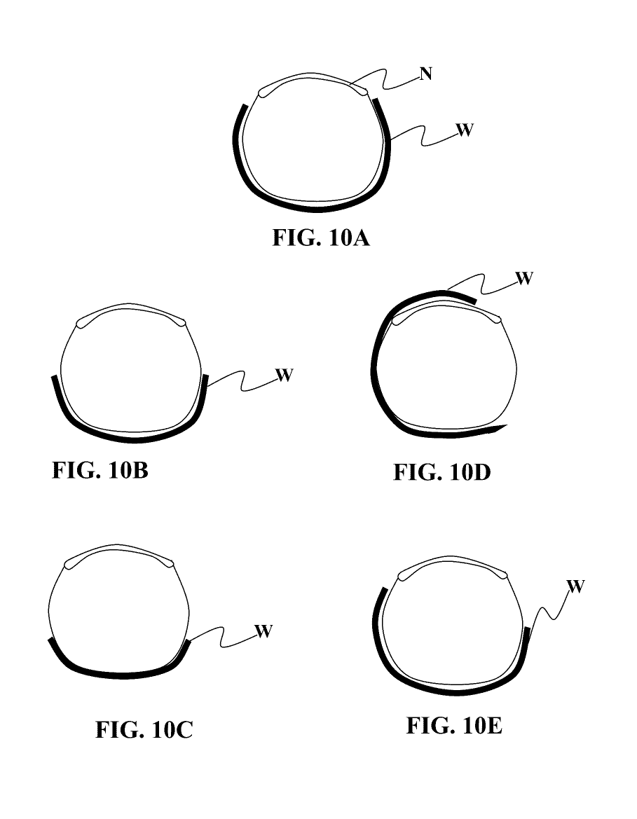

A further embodiment of devices for warming a fingertip, or for warming a plurality of fingers, or for warming a hand of a subject is shown in FIG. 9. The example shown in FIG. 9 illustrates such a warmer which utilizes warm air to warm a fingertip, or finger, or fingers, or a hand. A warmer as illustrated in FIG. 9 includes a heating element and a fan. A warmer as illustrated in FIG. 9 may also include a surface for resting a fingertip, finger, fingers, or hand of a subject; in embodiments, such a surface may be configured to guide or place a fingertip, finger, fingers, or hand of a subject into a proper or desired position or orientation for warming. As shown, the warmer may be configured to at least partially enclose a hand, or fingers, or a subject. In embodiments, a warmer using warm air to warm a finger or a fingertip may be configured to enclose a single finger, or to enclose a portion of a single finger (e.g., a fingertip) and to direct warm air towards, and onto, the fingertip, finger, fingers, or hand of the subject. An air-warmer having features a s disclosed herein may also include a screen, or a baffle, or vanes, or other elements to direct the flow of air in the desired direction. In addition, a screen, or a baffle, or vanes, or other elements may be effective to insure that the fingertip, or finger, or fingers, or hand of the subject does not approach too closely to the source of air (e.g., an orifice or vent); or to a fan or fan blades; or to a heating element; or other element or portion of the air-warmer to which contact with a subject is not desired. A pediatric subject can wear the warmer 20 or 100 in such an air warmer. In such a configuration, the heat providing object is outside and heat may come in through the thermally conductive portion 26, wherein any of the embodiments herein can be configured to have such a portion.

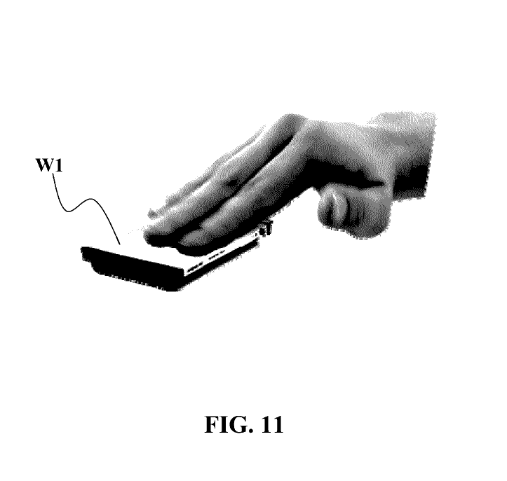

FIGS. 10A to 10E show the various areas that can be heated based on where the thermally conductive portion is located on the warmer W. FIGS. 10A to 10E show straight-on views of a finger with fingernail N with the warmer W wrapped fully or at least partially around the finger. For ease of illustration, the novelty item such as toy T is not shown in these figures, but it should be understood that they may house the warmer W and/or provided attachment to the subject's finger.

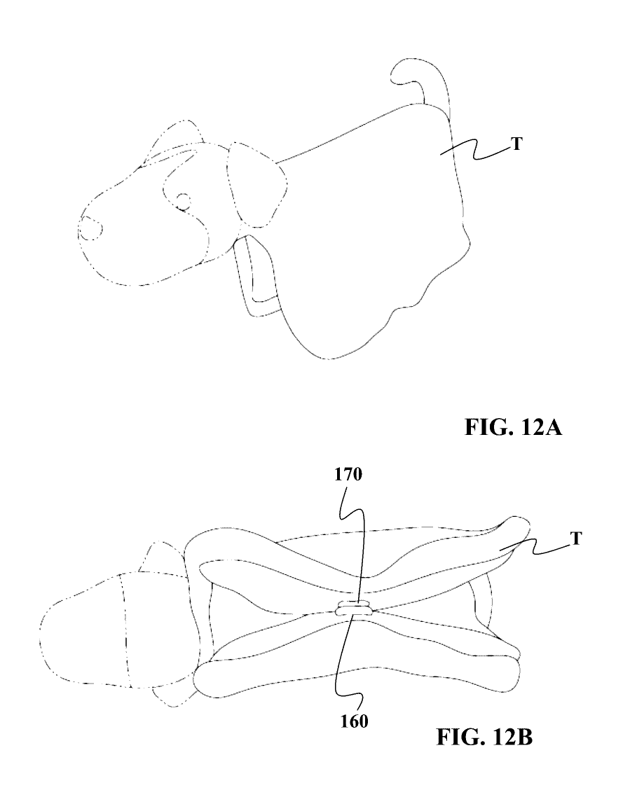

FIG. 11 shows that a warmer W1 for warming multiple fingers. A slightly different view of a warming plate in use warming fingers of a hand of a subject is shown in FIG. 11. A warming plate may be placed on, may be secured to, and may be embedded in, a table, desk, shelf, or other surface. In embodiments, a warming plate may be placed on, may be secured to, and may be embedded in, a surface effective to provide a convenient place for a subject to rest a finger, or fingers, or a hand, during warming. In embodiments, a warming plate may be placed on, may be secured to, and embedded in, the arm of a chair, sofa, couch, or other piece of furniture for seating effective to provide a convenient place for a subject to rest a finger, or fingers, or a hand, during warming.

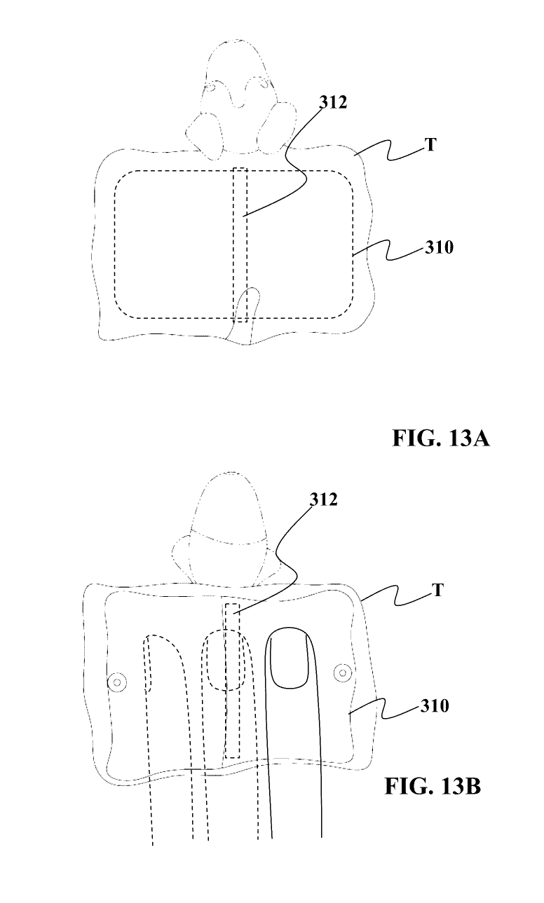

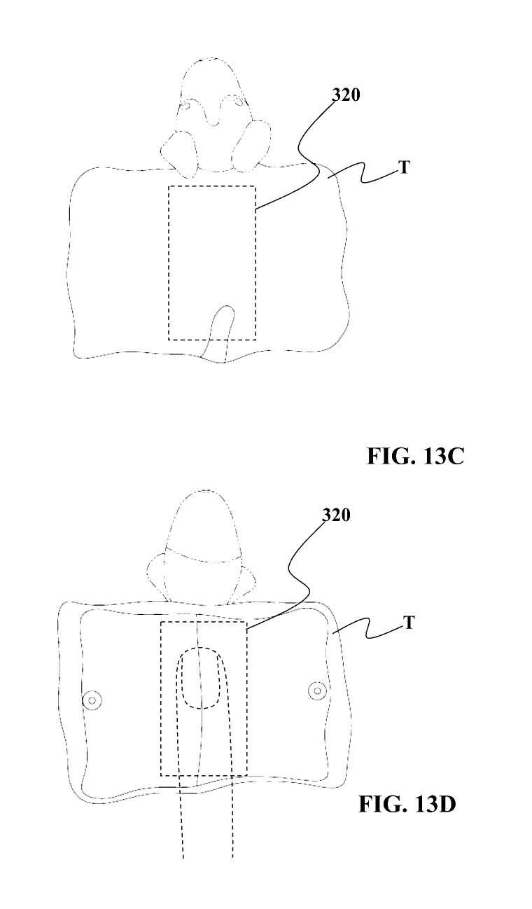

Following puncture of a finger (e.g., following a fingerstick), a subject, such as a child, may don a fingertoy for comfort, warmth, and amusement. An example of such a novelty item such as fingertoy T is shown in FIGS. 12A-13B. A fingertoy may be placed over a portion of the finger, such as a fingertip; for example, a fingertoy as disclosed herein may fit over a fingertip in a manner similar that is similar to the manner in which a thimble is commonly placed over a fingertip (or thumb-tip) of a person seeking to protect a fingertip during sewing. In embodiments, a fingertoy may be placed over a substantial portion of a finger, or over all of a finger, in a manner similar to the manner in which the finger of a glove covers a finger. A fingertoy may be made from, or may include (e.g., as an inner lining) an absorbent material. A fingertoy may be used in place of, or in conjunction with, a bandaid or other bandage. A fingertoy may include perfume or other odorant. A fingertoy T may keep a finger warm, or may provide compression, or may provide other physical sensations which may act to alleviate any discomfort which may ensue following a fingerstick. Optionally, some embodiments may use a novelty item such as a finger toy or plush toy to house a finger warmer to calm a pediatric subject in preparation, during, and/or after a sample collection process.

As shown, fingertoy may include features which may suggest a face, or arms, or hair, or other features which may be amusing, or diverting, or comforting, to a subject (e.g., a child) who has had a blood sample taken from a finger. A fingertoy may include writing, or symbols, or other markings which may provide amusement, diversion, or comfort, or which convey a message or reminder to a subject. In embodiments, a fingertoy may identify the location or enterprise which performed or collected the sample. In embodiments, a fingertoy may include a commercial message, or may include symbols or other markings which convey a commercial message.

FIG. 12B shows a finger warmer toy T may have fasteners 160 and 170. Fasteners 60 and 70 may be used to secure a finger warmer toy when the finger warmer and/or toy is rolled or folded around a finger. Fasteners 160 and 170 may be, for example, Velcro® fasteners which, when in contact with each other, are able to hold a finger warmer toy T in a rolled or folded configuration around a finger of a subject. Fasteners 160 and 170 may comprise different, complementary, fastener elements; for example where fasteners 160 and 170 comprise Velcro® fasteners, an external surface of fastener 160 differs from the corresponding, and complementary, surface of fastener 170. A further example in which fasteners160 and 170 comprise different fasteners is found where fasteners 160 and 170 form a snap, in which one of fasteners 160 and 170 has a protruding portion which fits snugly into a depression or receptacle in the other of fasteners 160 and 170. In embodiments, fastener 160 and fastener 170 may be similar, or may be identical; for example fastener 160 and fastener 170 may both comprise hooks configured to latch onto each other, or may comprise two portions of a zipper, or may comprise other fasteners. A fastener 160 may have a fastener width and a fastener length; a fastener 170 may have a fastener width and a fastener length. In the example shown in FIG. 12B, fastener width and fastener length are different than fastener width and fastener length; however, in embodiments, fastener width and fastener length may be the same as fastener width and fastener length. In embodiments, as shown in FIG. 12B, fasteners 160 and 170 may be placed along a midline (e.g., with centers along a line that is a distance from an outer edge of perimeter); placement along a midline as shown facilitates opposition of fastener 160 with fastener 170 and aids in their function of fastening to each other, effective to hold a finger warmer in a fastened configuration. In embodiments, so long as they may be manipulated so as to make contact and to fasten with each other, fasteners 160 and 170 may be placed asymmetrically. In embodiments, a finger warmer may have a strap, or belt, which may be used to hold a rolled or folded finger warmer in a rolled or folded configuration, e.g., around a finger.

Referring now to FIG. 13A, a top down view is shown of a finger toy T in an open configuration. As previously discussed, the toy T may be plush toy. Optionally, it may be a more hard-shell or other non-plush toy. FIG. 13A shows an outline 310 (shown in phantom) that may illustrate a pouch inside the toy T for holding a finger warmer. An opening 312 may be provided that allows for the insertion of the finger warmer. The opening 312 may be along the midline or other location of the toy T. It may be zippered, flapped, otherwise closeable, or just open. Although the toy T is shown as having a rectangular shape when viewed as shown in FIGS. 13A-13D, it should be understood that other shapes such as oval, round, polygonal, triangular, or single or multiple combinations of the foregoing. Some embodiments may have spherical, cubic, diamond, pyramid, or other shaped.

FIG. 13B shows an underside view of at least one of embodiments described herein. FIG. 13B shows various orientations and/locations that a user can place their finger in the warmer before the warmer is closed around the finger. For any of the embodiments herein, it should be understood that some embodiments may include at least one sanitary barrier layer that may be coupled to surface area of the toy T where a subject's finger may be held. In one embodiment, this layer remains in place even after use. Optionally, some embodiments may have the layer as removable layer that may be peeled away or otherwise detached so that the user is left is a novelty surface that was not exposed to user contact during the collection process. Coupling of the barrier to the toy may be by way of techniques currently known or that may be developed in the future. In one embodiment, this sanitary barrier may be sized to be similar to the area of pouch 310. Optionally, some may have the barrier on the top side and underside of the toy T. Optionally, some embodiments may cover the entire toy T with a barrier layer. It should also be understood that the side of toy T where the subject will place the finger may also have a reduced thickness material, such as but not limited to not having or reducing the amount of stuffing in that portion of the toy T.

Referring now to FIGS. 13C and 13D, the toy T maybe configured to hold a single finger warmer 320 that is covering only a reduced portion, such as but not limited to 50% or less of the surface on one side of the toy T. In one embodiment, an internal pouch 320 may be sized as shown in FIGS. 13C and 13D to be 50% or less of the surface on one side of the toy T. FIG. 13C shows that a pouch 320 may be accessible from a top side of the toy T while FIG. 13D shows that a pouch 320 may be accessible from a bottom side of the toy T. The opening 312 may be along the midline or other location of the toy T. It may be zippered, flapped, otherwise closeable, or just open. As seen in FIG. 13D, this configuration is conducive to have the subject place their finger in a central portion as shown in FIG. 13D. Again, such an embodiment may include a barrier layer as previously described herein. The barrier layer may optionally include text or images to calm the user and/or include instructions for the use of the finger warmer. After collection, the toy T may be given to the subject as a reward for their completion of the collection process. Some embodiments

Methods for obtaining a sample from a subject may comprise: providing a sample collection room in a retail store, the sample collection room including a warming table; and collecting a sample from a subject. Methods for obtaining a sample from a subject may comprise: providing a sample collection room in a retail store, the sample collection room including a warming plate; and collecting a sample from a subject. Applicants disclose methods for obtaining a sample from a subject, comprising providing a sample collection room in a retail store, the sample collection room including a warming plate; warming the subject's fingertip, or finger, or hand, and collecting a sample from a subject. In embodiments, the sample is a blood sample, e.g., a blood sample obtained from a fingerstick.

Methods for obtaining a sample from a subject may comprise: providing a sample collection room in a retail store, the sample collection room including a chair, bench, couch, or other piece of furniture for seating having a warming plate or other warming element (e.g., in an armrest); and collecting a sample from a subject. Applicants disclose methods for obtaining a sample from a subject, comprising providing a sample collection room in a retail store, the sample collection room including a chair, bench, couch, or other piece of furniture for seating having a warming plate or other warming element (e.g., in an armrest); warming the subject's fingertip, or finger, or hand, and collecting a sample from a subject. In embodiments, the sample is a blood sample, e.g., a blood sample obtained from a fingerstick.

Methods for obtaining a sample from a subject may comprise: providing a sample collection room in a retail store, the sample collection room including an air-warmer; and collecting a sample from a subject. Applicants disclose methods for obtaining a sample from a subject, comprising providing a sample collection room in a retail store, the sample collection room including an air-warmer; warming the subject's fingertip, or finger, or hand, and collecting a sample from a subject. In embodiments, the sample is a blood sample, e.g., a blood sample obtained from a fingerstick.

Methods for obtaining blood from a subject in a sample collection room may further include providing a post-draw toy to the subject (e.g., a child subject). For example, methods include obtaining blood from a subject in a sample collection room, using a warming plate and optionally providing a post-draw toy to the subject (e.g., a child subject). Methods may include obtaining blood from a subject in a sample collection room, using a finger-warmer and optionally providing a post-draw toy to the subject (e.g., a child subject). Methods may include obtaining blood from a subject in a sample collection room, using an air-warmer and optionally providing a post-draw toy to the subject (e.g., a child subject).

Applicants disclose methods for analyzing a sample obtained from a subject, comprising providing a sample collection room in a retail store; collecting a sample from a subject; and analyzing the sample. In embodiments, the sample collection room includes one or more of a warming table; a chair or other piece of furniture for seating having a warming plate or other warming element; an air-warmer; a calming feature; a fingertoy; and other elements. In embodiments, the sample collection room is adjacent to a bathroom. In embodiments, the sample collection room comprises a pass-through connecting to a bathroom adjacent to the sample collection room. In embodiments, the sample is analyzed at, or adjacent to, or near to, the sample collection room in the retail store. In embodiments, the sample is analyzed at a location other than at the retail store.

Accordingly, Applicants disclose methods for obtaining and analyzing a sample obtained from a subject, comprising: providing a sample collection room in a retail store; collecting a sample from a subject in the sample collection room; and analyzing the sample. Applicants disclose methods for obtaining and analyzing a sample obtained from a subject, comprising: providing a sample collection room in a retail store; collecting a small sample from a subject in the sample collection room; and analyzing the small sample. Applicants disclose methods for obtaining and analyzing a sample obtained from a subject, comprising: providing a sample collection room in a retail store; collecting a sample from a subject in the sample collection room; and analyzing the sample at the retail store location, or adjacent to the retail store location. In embodiments, these methods may include warming a body part of a subject prior to, or during, obtaining a sample from the body part. Such warming may include use of a warming table, a warming plate, an air-warmer, a fingertip warmer, a chair or other furniture for seating including a warming plate or other warming element, and combinations thereof.

A method for obtaining a sample from a subject, said subject having a body part, comprising: providing a sample collection room in a retail store; and obtaining a sample from said body part of said subject, wherein said obtaining is performed in said sample collection room. In embodiments, the methods comprise warming a body part; for example, a body part may be warmed prior to obtaining a sample from the body part, or may be warmed during obtaining a sample. In embodiments, warming of the body part comprises warming with a warming table. In embodiments, warming of the body part comprises warming with a warming plate. In embodiments, warming of the body part comprises warming with an air-warmer. In embodiments, the body part comprises a fingertip, a finger, a plurality of fingers, a hand, or other body part or body parts.

A method for analyzing a sample obtained from a subject, said subject having a body part, comprising: providing a sample collection room in a retail store; obtaining, in said sample collection room, a sample from said body part of said subject; and analyzing the sample at or adjacent to the retail store. In embodiments, the methods comprise warming a body part; for example, a body part may be warmed prior to obtaining a sample from the body part, or may be warmed during obtaining a sample. In embodiments, warming of the body part comprises warming with a warming table. In embodiments, warming of the body part comprises warming with a warming plate. In embodiments, warming of the body part comprises warming with an air-warmer. In embodiments, the body part comprises a fingertip, a finger, a plurality of fingers, a hand, or other body part or body parts.

A method for analyzing a sample obtained from a subject, said subject having a body part, comprising: providing a sample collection room in a retail store; obtaining, in said sample collection room, a sample from said body part of said subject; and analyzing the sample at or adjacent to the retail store within a short period of time after obtaining the sample. In embodiments, the methods comprise warming a body part; for example, a body part may be warmed prior to obtaining a sample from the body part, or may be warmed during obtaining a sample. In embodiments, warming of the body part comprises warming with a warming table. In embodiments, warming of the body part comprises warming with a warming plate. In embodiments, warming of the body part comprises warming with an air-warmer. In embodiments, the body part comprises a fingertip, a finger, a plurality of fingers, a hand, or other body part or body parts.

A method for analyzing a sample obtained from a subject, said subject having a body part, comprising: providing a sample collection room in a retail store; obtaining, in said sample collection room, a sample from said body part of said subject; transporting the sample to an analysis location; and analyzing the sample at said analysis location within a short period of time after obtaining the sample. In embodiments, the methods comprise warming a body part; for example, a body part may be warmed prior to obtaining a sample from the body part, or may be warmed during obtaining a sample. In embodiments, warming of the body part comprises warming with a warming table. In embodiments, warming of the body part comprises warming with a warming plate. In embodiments, warming of the body part comprises warming with an air-warmer. In embodiments, the body part comprises a fingertip, a finger, a plurality of fingers, a hand, or other body part or body parts.

A sample obtained by such methods may be a small sample. For example, such a sample may be obtained from a finger-stick, and may comprise a few drops, or two drops, or one drop, of blood obtained from a small lancet puncture in the skin of the subject. The sample, such as a blood sample, may be obtained from the subject following warming a body part (e.g., a fingertip, or finger, of fingers, or hand) of subject. For example, in embodiments, the sample is a small volume sample of blood, or of urine, or of saliva, or of tears, or other bodily secretion or excretion. For example, in embodiments, the sample is a small sample having a volume of less than about 200 μL, or less than about 150 μL, or less than about 100 μL, or less than about 75 μL, or less than about 50 μL, or less than about 25 μL, or less.

In embodiments, a sample collection room includes a location for a practitioner (e.g., a sample collection technician) to sit and obtain a bodily fluid or other sample from the patient. A work surface such as a table is provided so that a patient has a surface on which they can position the target site on the subject to allow for sampling.

The work surface may have one or more thermal controlled sites wherein the temperature of the target site on the patient may be brought to a desired temperature. The temperature may be adjustable. By way of example and not limitation, embodiments may heat a finger or other target site to improve blood flow and thus blood yield from a finger-stick. For example, the table may have thermal control areas to increase blood flow to the target area and thus increase the speed with which sufficient blood or other bodily fluid can be drawn from the subject. The heating is used to bring the target tissue to between about 40° C. to about 50° C. In embodiments, the heating brings target tissue to within a temperature range of between about 40° C. to about 44° C. In embodiments, the heating brings target tissue to within a temperature range of between about 41° C. to about 43° C. In embodiments, the heating brings target tissue to a temperature of about 42° C. In embodiments, the heating brings target tissue to within a temperature range of between about 44° C. to about 47° C. In one embodiment, the temperature is sufficient to increase blood flow to yield 120 uL of sample. In one embodiment, the temperature is sufficient to increase blood flow to yield 130 uL of sample. In one embodiment, the temperature is sufficient to increase blood flow to yield 140 uL of sample. In one embodiment, the temperature is sufficient to increase blood flow to yield 150 uL of sample. Optionally, the thermal controlled site is a shaped surface is contoured to match that of the target site on a patient.

In embodiments, the thermal controlled site comprises a hard surface that is easily cleaned, and that may be sterilized. Such a surface may be or include, for example, plastic, glass, hard rubber, acrylic, polymer, and other materials. In embodiments, the thermal controlled site comprises a soft or padded surface that is easily cleaned, and that may be sterilized. Such a surface may be or include, for example, cloth, rubberized cloth, soft rubber, and other materials.

In embodiments, a warming table is configured to place the hand or other body part from which a blood sample is to be collected at an optimal height and orientation for facilitating the flow of gravity in the finger as blood droplets form. A warming table may include a rest, such as an arm rest, or hand rest, or other rest which provides an additional surface for contacting a subject; such a surface may make positioning of a hand, or arm, or foot, or leg, other body part more comfortable for the subject, more convenient for the sample collection technician, or both. Such a rest may be configured for placement in more than one position or orientation. Such a rest may be retractable or otherwise configured to be placed or stored out of the way of a subject or sample collection technician when not needed. A warming table may be designed to accommodate the seating or placement of a sample collection technician near to a subject. A warming table, or surface thereof, may be adjustable, or may include an adjustable surface, which may be placed or oriented for the comfort and convenience of a subject, a sample collection technician, or both. For example, a warming table may include a sliding surface which may be extended or retracted as needed to accommodate a subject or a sample collection technician; may include a tiltable surface which may be oriented as needed to accommodate a subject or a sample collection technician; may include a surface which may be raised or lowered as needed to accommodate a subject or a sample collection technician; or be otherwise adjustable.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, with any of the above embodiments, it should be understood that although some embodiments may refer to the warmer as a fingertip warmer, some embodiments can heat other portions of the finger or have an opening at a distal end that allows some portion of the fingertip to extend outside of the warmer. In this manner, warmers are not limited to only fingertip warmers. It should also be understood that although most of the embodiments herein discuss heating the target tissue, it should also be understood that the system can also be used for other thermal conditioning such as but not limited to cooling. Some embodiments may have a cooling object in one pouch and a heating element in another pouch. Some embodiments may use variations of heating and cooling, warm and warmer zones, cool and cooler zones, or other configurations to tailor custom thermal profiles at or near a target tissue. It should also be understood that in some embodiments, thermal element is sized to be larger than the pouch so that at least a portion extends beyond the pouch. This can be useful for ease of removal and/or for increasing the amount of area being treated. Although many embodiments are described in the context of warming a fingertip, it should understood that the warmer can be used heat the palm or other surfaces of the hand. Optionally, some embodiments may configure it heat a heel or other tissue area on the subject. Some embodiments may have a method where the subject is given the toy on a hand that is not going to be used for collection, so as to distract the subject. Optionally, some may have a method where the subject is given at least two of the toys to distract them simultaneously. Some embodiments may include LED lights, EL panels, sound making electronics, talking electronics, or the like to increase the calming and/or distracting effect. Although the toy portion of the novelty item is shown in the figures as a dog or jellyfish, it should understood that other animals are not excluded. Some of the toy portion may be plush, such as stuffed with a cotton or other filling, while some or all of other portions are non-plush such as but not limited to plastic, rubber, or polymer. Some embodiments may have representations of cars, trucks, fire engines, ships, trains, airplanes, rockets, submarines, rocks, cells, biological organisms, cartoon characters, movie characters, sports figures, trophies, awards, or any number of other items that may serve as an interest item to the subject during the collection process.

Additionally, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a size range of about 1 nm to about 200 nm should be interpreted to include not only the explicitly recited limits of about 1 nm and about 200 nm, but also to include individual sizes such as 2 nm, 3 nm, 4 nm, and sub-ranges such as 10 nm to 50 nm, 20 nm to 100 nm, etc. . . .

The publications discussed or cited herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. All publications mentioned herein are incorporated herein by reference to disclose and describe the structures and/or methods in connection with which the publications are cited. The following patent applications are fully incorporated herein by reference for all purposes: PCT/US14/54625 filed Sep. 8, 2014, U.S. Patent App. Ser. No. 61/894,166 filed Oct. 22, 2013 and U.S. Patent App. Ser. No. 61/890,870 filed Oct. 14, 2013.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. Any feature, whether preferred or not, may be combined with any other feature, whether preferred or not. The appended claims are not to be interpreted as including means-plus-function limitations, unless such a limitation is explicitly recited in a given claim using the phrase “means for.” It should be understood that as used in the description herein and throughout the claims that follow, the meaning of “a,” “an,” and “the” includes plural reference unless the context clearly dictates otherwise. For example, a reference to “an assay” may refer to a single assay or multiple assays. Also, as used in the description herein and throughout the claims that follow, the meaning of “in” includes “in” and “on” unless the context clearly dictates otherwise. Finally, as used in the description herein and throughout the claims that follow, the meaning of “or” includes both the conjunctive and disjunctive unless the context expressly dictates otherwise. Thus, the term “or” includes “and/or” unless the context expressly dictates otherwise.

This document contains material subject to copyright protection. The copyright owner (Applicant herein) has no objection to facsimile reproduction of the patent documents and disclosures, as they appear in the US Patent and Trademark Office patent file or records, but otherwise reserves all copyright rights whatsoever. The following notice shall apply: Copyright 2013-2014 Thermos, Inc.

1. (canceled).

2. (canceled).

3. A combination toy and finger warmer comprising:
a finger warmer device;
a plurality of structural elements connectable to one another in a surrounding arrangement relative to the finger warmer device; where the plurality of structural elements comprise at least a plurality of first connector portions and a plurality of second connector portions, where all of the first connector portions have a first form and all of the second connector portions have a second form, such that any number of the structural elements can be joined with or without the warmer to form a toy assembly.

4. (canceled)

5. A method for analyzing a sample obtained from a subject, said subject having a body part, comprising:
providing a sample collection room in a retail store;
warming a target tissue area on a subject with a combination novelty toy and warming device;
obtaining, in said sample collection room, a sample from said body part of said subject;
transporting the sample to an analysis location; and
analyzing the sample at said analysis location within a short period of time after obtaining the sample.

6. The method of claim 2 further comprising giving only the novelty toy with holder, but not the warming device, to the subject after the collection.

7. The method of claim 5, wherein said warming of said body part comprises warming with a warming table; a warming plate; a fingertip warmer; an air-warmer; furniture for seating comprising a warming plate or other heating element; or a combination thereof.

8. The method of claim 5 wherein the warming device is selected from a warming table, a warming plate, a chair or other furniture for seating comprising a warming plate, a fingertip warmer, and an air-warmer.

9. The method of claim 5 wherein said warming device is configured to warm a body part of a subject effective to aid in the collection of a blood sample from said body part.

10. The method of claim 5 wherein said target tissue is selected from a fingertip, a finger, and a hand.

11-16. (canceled)

17. A novelty item for use with a warming device, the novelty item comprising:
at least one structural element defining at least one cavity for receiving the warming device, wherein the cavity is positioned within said novelty item to position the warming device beneath a surface of the novelty item that is configured to be in contact with at least a portion of a subject being treated; at least a second structural element defining a toy portion of the novelty item.

18. The novelty of claim 17 wherein said toy portion resembles a stuffed animal.

19. The novelty of claim 17 wherein said toy portion resembles a toy dog.

20. The novelty of claim 17 wherein said warming device comprises a sealed pouch comprising a solution of supersaturated sodium acetate in water and a trigger element.
